SEQUENCE LISTING (2 sequences)

SEQ ID NO: 1 - DNA, 15 bp, Artificial Sequence
  Description of Artificial Sequence oligonucleotide
ccctcaagcagagga

SEQ ID NO: 2 - DNA, 15 bp, Artificial Sequence
  Description of Artificial Sequence oligonucleotide
tcctctgcttgaggg

(19) United States
(12) Patent Application Publication
(10) Pub. No.: US 2002/0051975 A1
(43) Pub. Date: May 2, 2002

(54) REPORTERLESS GENOSENSORS USING ELECTRICAL DETECTION METHODS

(76) Inventors: CHANGMING LI, PHOENIX, AZ (US); SONG SHI, PHOENIX, AZ (US); GEORGE MARACAS, PHOENIX, AZ (US); VI-EN CHOONG, CHANDLER, AZ (US)

Correspondence Address: ROBIN M. SILVA, ESQ., FLEHR HOHBACH TEST ALBRITTON AND HERBERT, LLP, FOUR EMBARCADERO CENTER, SUITE 3400, SAN FRANCISCO, CA, 94111, US

(21) Appl. No.: 09/458,533

(22) Filed: Dec. 9, 1999

(51) Int. Cl. C12Q 1/68; C07H 21/04; C12P 19/34; C07H 21/02

(52) U.S. Cl. 435/6; 435/91.1; 435/91.2; 536/23.1

(57) ABSTRACT

The present invention provides an apparatus and methods for detecting cation interactions associated with molecular interactions using AC impedance, but without the use of electrochemical or other reporters to obtain measurable signals. The methods can be used for electrical detection of molecular interactions between probe molecules bound to defined regions of an array and target molecules which are permitted to interact with the probe molecules.

### BACKGROUND OF THE INVENTION

1. Field of the Invention

This invention relates to the detection of molecular interactions between biological molecules. Specifically, the invention relates to electrical detection of interactions such as hybridization between nucleic acids or peptide antigen-antibody interactions using arrays of peptides or oligonucleotides. In particular, the invention relates to an apparatus and methods for detecting nucleic acid hybridization or peptide binding using AC impedance, but without requiring the use of electrochemical or other reporter moieties to obtain measurable signals.

2. Background of the Invention

A number of commonly-utilized biological applications, including for example, diagnoses of genetic disease, analyses of sequence polymorphisms, and studies of receptor-ligand interactions, rely on the ability of analytical technologies to readily detect events related to the interaction between probe and target molecules. While these molecular detection technologies have traditionally utilized radioactive isotopes or fluorescent compounds to monitor probe-target interactions, methods for the electrical detection of molecular interactions have provided an attractive alternative to detection techniques relying on radioactive or fluorescent labels.

Electrical and electrochemical detection techniques are based on the detection of alterations in the electrical properties of an electrode arising from interactions between probe molecules on the surface of the electrode and target molecules in the reaction mixture. Electrical or electrochemical detection eliminates many of the disadvantages inherent in use of radioactive or fluorescent labels to discern molecular interactions. This process offers, for example, a detection technique that is safe, inexpensive, and sensitive, and is not burdened with complex and onerous regulatory requirements.

However, despite these advantages, there are a number of obstacles in using electrical or electrochemical detection techniques for analyzing molecular interactions. One such obstacle is the requirement, in some methods, of incorporating an electrochemical label into the target molecule. For example, labeled target molecules have been used to increase the signal produced upon the formation of nucleic acid duplexes during hybridization assays.

For example, Meade et al (in U.S. Pat. Nos. 5,591,578, 5,705,348, 5,770,369, 5,780,234 and 5,824,473) provide methods for the selective covalent modification of nucleic acids with redox-active moieties such as transition metal complexes. Meade et al. further disclose nucleic acid hybridization assays employing such covalently-modified nucleic acid molecules.

Heller et al. (in U.S. Pat. Nos. 5,605,662 and 5,632,957) provide methods for controlling molecular biological reactions in microscopic formats that utilize a self-addressable, self-assembling microelectronic apparatus. Heller et al. further provide an apparatus in which target molecules labeled with fluorescent dyes are transported by free field electrophoresis to specific test sites where the target molecules are concentrated thereby, and reacted with specific probes bound to that test site. Unbound or non-specifically interacting target molecules are thereafter removed by reversing the electric polarity at the test site. Interactions between probe and target molecules are subsequently assayed using optical means.

Certain alternative methods that do not employ labeled target nucleic acids have been described in the prior art. For example, Hollis et al. (in U.S. Pat. Nos. 5,653,939 and 5,846,708) provide a method and apparatus for identifying molecular structures within a sample substance using a monolithic array of test sites formed on a substrate upon which the sample substance is applied. In the method of Hollis et al., changes in the electromagnetic or acoustic properties—for example, the change in resonant frequency—of the test sites following the addition of the sample substance are detected in order to determine which probes have interacted with target molecules in the sample substance.

In addition, Eggers et al. (in U.S. Pat. Nos. 5,532,128, 5,670,322, and 5,891,630) provide a method and apparatus for identifying molecular structures within a sample substance. In the method of Eggers et al., a plurality of test sites to which probes have been bound is exposed to a sample substance and then an electrical signal is applied to the test sites. The dielectrical properties of the test sites are subsequently detected to determine which probes have interacted with target molecules in the sample substance.

Another obstacle in the development of a simple and cost-effective electrical and electrochemical detection apparatus for detecting molecular interactions involves the attachment of probe molecules to the microelectrodes or substrate of a microarray. For example, although the prior art provides microarrays using polyacrylamide pads for attachment of oligonucleotide probes to a solid support, the art has not provided such pads in conjunction with an electrical or electrochemical detection apparatus.

Guschin et al., 1997, Anal. Biochem. 250: 203-11 describe a technique for detecting molecular interactions between target molecules in a biological sample solution and polyacrylamide gel-immobilized probes on a glass substrate. In the technique disclosed by Guschin et al., molecular interactions between probes and target molecules are detected using optical reporters. The Guschin et al. reference neither teaches nor suggests using electrical or electrochemical detection techniques to detect hybridization between target molecules and immobilized probes.

Guschin et al., 1997, Appl. Environ. Microbiol. 63: 2397-402 also describe the fabrication of microarrays through the immobilization of oligonucleotide probes on a polyacrylamide gel pad placed in contact with a glass substrate. In this technique disclosed by Guschin et al., parallel hybridization between target nucleic acids and immobilized probes is detected using optical reporter moieties. This Guschin et al. reference also does not teach or suggest using electrical or electrochemical detection techniques in combination with the immobilization of probes on polyacrylamide gel pads.

In addition, Yang et al., 1997, Anal. Chim. Acta 346: 259-75 describe the fabrication of microarrays through the immobilization of nucleic acid probes on polyacrylamide gel pads and subsequent detection of molecular interactions between probe and target molecules using optical reporter moieties. Yang et al. further describe an alternative technique in which molecular interactions between labeled target molecules and nucleic acid probes that have been directly attached to solid electrodes are detected using electrical or electrochemical means. Yang et al., however, does not suggest using electrical or electrochemical detection techniques in combination with the immobilization of probes on polyacrylamide gel pads.

There remains a need in the art to develop alternatives to current detection methods used to detect interactions between biological molecules, particularly nucleic acids and peptides. In particular, there is a need in the art to develop electrical or electrochemical methods for detecting interactions between biological molecules that do not require modifying target or probe molecules with reporter labels. The development of such methods has wide applications in the medical, genetic, and molecular biological arts. There further remains a need in the art to develop alternatives for the attaching such biological molecules to the microelectrodes or substrate of an electrical or electrochemical device.

### SUMMARY OF THE INVENTION

The present invention provides an apparatus and methods, using cations in an electrolyte solution, for detecting the nature and extent of molecular interactions between probe and target molecules. The most preferred embodiments of the methods of the invention utilize AC impedance for said detection. The apparatus and methods of the present invention have the advantage of providing electrical detection without any additional requirement that the target molecule be labeled with a reporter molecule.

In preferred embodiments of the present invention, the apparatus and methods are useful for detecting molecular interactions such as nucleic acid hybridization between oligonucleotide probe molecules bound to defined regions of an ordered array and nucleic acid target molecules which are permitted to interact with the probe molecules. In other embodiments of the present invention, the apparatus and methods are useful for detecting interactions between peptides (e.g., receptor-ligand binding or antibody recognition of antigens).

In more preferred embodiments, the apparatus of the present invention comprises a supporting substrate, an array of microelectrodes in contact with the supporting substrate to which probes are immobilized, at least one counter-electrode in electrochemical contact with the supporting substrate, a means for producing electrical impedance at each microelectrode, a means for detecting changes in impedance at each microelectrode in the presence or absence of a target molecule, and an electrolyte solution in contact with the plurality of microelectrodes.

In alternative preferred embodiments, the apparatus of the present invention comprises a supporting substrate, an array of microelectrodes in contact with the supporting substrate, a plurality of polyacrylamide gel pads in contact with microelectrodes and to which probes are immobilized, at least one counter-electrode in electrochemical contact with the supporting substrate, a means for producing electrical impedance at each microelectrode, a means for detecting changes in impedance at each microelectrode in the presence or absence of a target molecule, and an electrolyte solution in contact with the plurality of microelectrodes. Alternatively, multiple electrodes can be defined on a substrate and covered with a continuous, unpatterned layer of polyacrylamide or other polymer.

In preferred embodiments of the present invention, microelectrodes are prepared from metals such as dense or porous films of gold, platinum, titanium, or copper, metal oxides, metal nitrides, metal carbides, or carbon.

In some embodiments of the present invention, the probes are oligonucleotide probes having a sequence comprising from about 10 to about 30 nucleotide residues wherein said probes are attached to a conjugated polymer or copolymer film that is in contact with the microelectrodes. The conjugated polymer or copolymer film used for probe attachment includes, but is not limited to, polypyrrole, polythiphene, polyaniline, polyfuran, polypyridine, polycarbazole, polyphenylene, poly(phenylenvinylene), polyfluorene, polyindole, their derivatives, their copolymers, and combinations thereof. In a preferred embodiment, the oligonucleotide probes are attached to the microelectrodes through a neutral polypyrrole matrix.

In other embodiments of the present invention, the probes are oligonucleotide probes having a sequence comprising from about 10 to about 30 nucleotide residues and said probes are attached to polyacrylamide gel pads that are in contact with the microelectrodes.

In still other embodiments, the probes are peptides, such as receptors, ligands, antibodies, antigens, or synthetic peptides, and said probes are attached to the microelectrodes or polyacrylamide gel pads using techniques known to those with skill in the art.

In a preferred embodiment of the invention, the electrolyte solution comprises metal cations or polymerized cations that are ion conductive and capable of reacting with probes or probe-target complexes. In a more preferred embodiment, the electrolyte solution comprises a salt of a lithium cation, most preferably LiClO₄.

The apparatus of the present invention may further comprise at least one reference electrode. In an alternative embodiment of the present invention, the apparatus further comprises a plurality of wells each of which encompasses at least one microelectrode and at least one counter-electrode that is sufficient to interrogate the entire array.

In a preferred method of the present invention, an electrolyte solution as described above is placed in contact with a plurality of microelectrodes to which nucleic acid probes have been immobilized, preferably having a neutral polypyrrole layer there between. AC impedance of the microelectrodes is first measured in the absence of added target nucleic acid. Thereafter, the microelectrodes are contacted with a biological sample substance containing target nucleic acid molecules, most preferably by adding the sample to the electrolyte solution or replacing the electrolyte solution with the sample contained in or diluted in the electrolyte solution. The probes and target molecules are allowed to interact, preferably by hybridization, and the AC impedance measured thereafter.

In another embodiment of the methods of the present invention, an electrolyte solution as described above is placed in contact with a plurality of microelectrodes and polyacrylamide gel pads to which nucleic acid probes have been immobilized. AC impedance of the microelectrodes is first measured in the absence of added target nucleic acid. Thereafter, the microelectrodes are contacted with a biological sample substance containing target nucleic acid molecules, most preferably by adding the sample to the electrolyte solution or replacing the electrolyte solution with the sample contained in or diluted in the electrolyte solution. The probes and target molecules are allowed to interact, preferably by hybridization, and the AC impedance measured thereafter.

In a preferred embodiment of the methods of the present invention, the AC impedance is measured at different frequencies in order to increase the sensitivity of the method. Probe-target interactions are detected by differences in the AC impedance signals at individual microelectrodes before and after such interactions. Most preferably, the method is used to discern the difference between hybridization between an immobilized oligonucleotide probe on a microelectrode and a complimentary target nucleic acid (“complete” hybridization), and hybridization between the immobilized oligonucleotide and a noncomplementary target nucleic acid (“noncomplementary” hybridization). Information about the nucleotide sequence of the oligonucleotides immobilized at each microelectrode is then used in conjunction with “complete” or “mismatch” hybridization as detected by the method of the invention to determine the presence or absence of a particular target nucleic acid in the sample.

Specific preferred embodiments of the present invention will become evident from the following more detailed description of certain preferred embodiments and the claims.

### DESCRIPTION OF THE DRAWINGS

FIGS. 1A and 1B illustrate a schematic representation of the structure of a hydrogel porous microelectrode (FIG. 1A) and a schematic representation of the structure of the tip of a hydrogel porous microelectrode (FIG. 1B);

FIGS. 2A and 2B illustrate the electrochemical oxidation of pyrrole (FIG. 2A) and the neutralization of polypyrrole (FIG. 2B);

FIGS. 3A and 3B illustrate the Frequency Complex curves obtained from polypyrrole microelectrodes before and after the hybridization of a 15-mer oligonucleotide probe and complementary nucleic acid target molecule (FIG. 3A) and the Frequency Complex curve obtained in the high frequency zone from polypyrrole microelectrodes before and after the hybridization of a 15-mer oligonucleotide probe and complementary target molecule (FIG. 3B);

FIGS. 4A and 4B illustrate a plot of low frequency resistance versus ω^{-½} (FIG. 4A) and the plot of high frequency resistance versus ω^{-½} (FIG. 4B);

FIGS. 5A and 5B illustrate the Frequency Complex curve obtained for the hybridization of an oligonucleotide probe and a fully complementary nucleic acid target molecule (FIG. 5A) and the Frequency Complex curve obtained for the hybridization of an oligonucleotide probe and a nucleic acid target molecule possessing three mismatches (FIG. 5B; curve 1 was obtained before hybridization of the target molecule to the probe, curve 2 was obtained following hybridization of probe and target molecules for 48 hours, curve 3 was obtained following washing of hybridized molecules for 30 min. at 37° C., and curve 4 was obtained following washing of hybridized molecules for 30 min. at 38° C.);

FIG. 6 illustrates a plot of low frequency resistance versus ω^{-½} obtained for the hybridization of an oligonucleotide probe and a nucleic acid target molecule possessing three mismatches (curve 1 was obtained before hybridization of the target molecule to the probe, curve 2 was obtained following hybridization of probe and target molecules for 48 hours, curve 3 was obtained following washing of hybridized molecules for 30 min. at 37° C., and curve 4 was obtained following washing of hybridized molecules for 30 min. at 38° C.)

FIG. 7 illustrates the Frequency Complex curve obtained from polypyrrole microelectrodes before and after the hybridization of a 15-mer oligonucleotide probe and complementary nucleic acid target molecule in an electrolyte containing 0.1 M LiClO₄;

FIGS. 8A through 8C illustrate a schematic representation of the circuit (FIG. 8A), the AC impedance response for a polypyrrole microelectrode with an attached single-strand nucleic acid probe before hybridization to a target molecule (FIG. 8B), and a schematic representation of the circuit for a polypyrrole microelectrode with an attached single-strand nucleic acid probe after hybridization to a target molecule (FIG. 8C);

FIG. 9 illustrates a plot of capacitance versus frequency for a polypyrrole microelectrode with an attached single-strand nucleic acid probe after hybridization to a target molecule;

FIG. 10 illustrates a plot of resistance versus frequency for a polypyrrole microelectrode with an attached single-strand nucleic acid probe after hybridization to a target molecule;

FIG. 11 illustrates a hydrogel porous microelectrode;

FIG. 12 illustrates the Frequency Complex curves obtained from a hydrogel porous microelectrode with attached 15-mer oligonucleotide probe in the absence of a complementary target molecule (curve 1), following incubation with 2 pM of a complementary target molecule (curve 2), and following incubation with 300 nM of a noncomplementary target molecule (curve 3);

FIG. 13 illustrates a plot of capacitance versus frequency for a hydrogel porous microelectrode with an attached single-strand nucleic acid probe after hybridization to a target molecule;

FIG. 14 illustrates a plot of resistance versus frequency for a hydrogel porous microelectrode with an attached single-strand nucleic acid probe after hybridization to a target molecule.

### DETAILED DESCRIPTION OF THE PREFERRED EMBODIMENTS

The invention provides an apparatus and methods for using the apparatus to determine the presence or absence of a target molecule, most preferably a nucleic acid, in a biological sample. Alternatively, the invention provides an apparatus and methods for using the apparatus to determine the presence or absence of a target peptide or polypeptide in a biological sample.

The apparatus and methods of the present invention are illustrated herein using hybridization between oligonucleotide probes immobilized on microelectrodes and target nucleic acid molecules contained in a biological sample. The phosphate groups of nucleic acids are negatively charged at all biologically relevant pH values. Thus, a nucleic acid duplex possesses a high negative charge density. Following electrical perturbation of the nucleic acid, strong interactions, such as the intercalation or binding of metal ions to the nucleic acid, occur. These interactions are dependent upon the structure and charge density of the nucleic acid. Since the structural and electrical properties of a nucleic acid molecule (such as a probe) are altered when the probe is hybridized to a suitable target molecule, the result of this molecular interaction is a change in AC impedance. This change is used in the methods and apparatus of the invention to distinguish between “complete” hybridization and incomplete or “mismatch” hybridization between the immobilized oligonucleotide probe and target nucleic acid.

In one embodiment, the apparatus of the present invention comprises a supporting substrate, a plurality of microelectrodes in contact with the supporting substrate to which probes are immobilized, at least one counter-electrode in contact with the supporting substrate, a means for producing electrical impedance at each microelectrode, a means for detecting changes in impedance at each microelectrode in the presence or absence of a target molecule, and an electrolyte solution in contact with the plurality of micro electrodes.

In another embodiment, the apparatus of the present invention comprises a supporting substrate, a plurality of microelectrodes in contact with the supporting substrate, a plurality of polyacrylamide gel pads in contact with the microelectrodes and to which probes are immobilized, at least one counter-electrode in contact with the supporting substrate, a means for producing electrical impedance at each microelectrode, a means for detecting changes in impedance at each microelectrode in the presence or absence of a target molecule, and an electrolyte solution in contact with the plurality of micro electrodes.

In one embodiment, the apparatus is a microarray containing at least 5 microelectrodes on a single substrate to which oligonucleotide probes have been attached. Alternatively, arrayed oligonucleotides are attached to polyacrylamide gel pads that are in contact with the microelectrodes of the apparatus of the present invention. Most preferably, oligonucleotides having a particular nucleotide sequence, or groups of such oligonucleotides having related (e.g., overlapping) nucleotide sequences, are immobilized at each of the plurality of microelectrodes. In further preferred embodiments, the nucleotide sequence(s) of the immobilized oligonucleotides at each microelectrode, and the identity and correspondence between a particular microelectrode and the nucleotide sequence of the oligonucleotide immobilized thereto, are known.

In preferred embodiments, the probes are oligonucleotides comprising from about 10 to about 100, more preferably from about 10 to about 50, and most preferably from about 15 to about 30, nucleotide residues. In alternative embodiments, the probes are nucleic acids comprising from about 10 to about 5000 basepairs, more preferably from about 100 to about 1000 basepairs, and most preferably from about 200 to about 500 basepairs. In further preferred embodiments, the immobilized probes are peptides comprising from about 5 to about 500 amino acid residues.

In the preferred embodiment of the apparatus of the present invention, the substrate is composed of silicon. In alternative embodiments, the substrate is prepared from substances including, but not limited to, glass, plastic, rubber, fabric, or ceramics. The microelectrodes are embedded within or placed in contact with the substrate.

In preferred embodiments, microelectrodes are prepared from substances including, but not limited to, metals such as gold, silver, platinum, titanium or copper, in solid or porous form and preferably as foils or films, metal oxides, metal nitrides, metal carbides, or carbon. In certain preferred embodiments, probes are attached to a conjugated polymer or copolymer film including, but not limited to, polypyrrole, polythiphene, polyaniline, polyfuran, polypyridine, polycarbazole, polyphenylene, poly(phenylenvinylene), polyfluorene, polyindole, their derivatives, their copolymers, and combinations thereof. In alternative embodiments, probes are attached to polyacrylamide gel pads that are in contact with the microelectrodes.

The substrate of the present invention has a surface area of between 0.01 μm² and 5 cm² containing between 1 and 1×10⁸ microelectrodes. In one embodiment, the substrate has a surface area of 100 μm² and contains 10⁴ microelectrodes, each microelectrode having an oligonucleotide having a particular sequence immobilized thereto. In another embodiment, the substrate has a surface area of 100 μm² and contains 10⁴ microelectrodes, each microelectrode in contact with a polyacrylamide gel pad to which an oligonucleotide having a particular sequence has been immobilized thereto. In preferred embodiments, the microelectrodes are arranged on the substrate so as to be separated by a distance of between 0.05 μm to 0.5 mm. Most preferably, the microelectrodes are regularly spaced on the solid substrate with a uniform spacing there between.

In some embodiments of the present invention, the microelectrodes project from the surface of the substrate, with such projections extending between 5×10⁻⁸ and 1×10⁻⁵ cm from the surface of the substrate. In other embodinents, the microelectrodes comprise a flat disk of conductive material that is embedded in the substrate and exposed at the substrate surface, with the substrate acting as an insulator in the spaces between the microelectrodes.

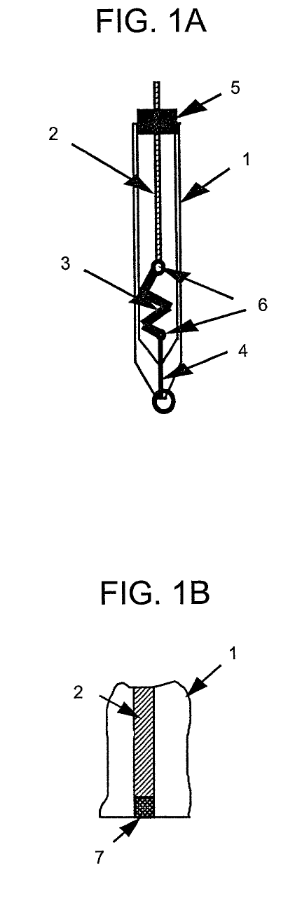

In the preferred embodiment of the present invention the microelectrodes comprise a gold conductor and glass insulator. In alternative embodiments, the microelectrodes comprise conductor substances such as solid or porous films of silver, platinum, titanium, coppers or metal oxides, metal nitrides, metal carbides, or carbon (graphite). In alternative embodiments, the microelectrodes comprise substrate and/or insulator substances such as glass, silicon, plastic, rubber, fabric, or ceramics. The microelectrodes of the present invention have an exposed conductive surface of between 0.01 μm² to 0.5 cm². In the preferred embodiment, the exposed conductive material is between 100 to 10,000 μm². One embodiment of the present invention is shown in FIG. 1A, wherein the microelectrode comprises a glass capillary tube 1, containing an ultra fine platinum wire 2, to which a transition wire 3 has been soldered 6. The transition wire 3, is soldered 6 in turn to a hookup wire 4, which protrudes from an epoxy plug 5 that seals the capillary tube. In one embodiment of the present invention, polyacrylamide gel material 7 is packed into a recess etched into the exposed surface of the platinum wire 2.

In some embodiments, oligonucleotide probes are immobilized on the microelectrodes of the apparatus of the present invention using a neutral layer between the oligonucleotides and the microelectrodes. In a preferred embodiment, this layer comprises neutral polypyrrole. In alternative embodiments, this layer comprises such substances as polythiphene, polyaniline, polyfuran, polypyridine, polycarbazole, polyphenylene, poly(phenylenvinylene), polyfluorene, polyindole, their derivatives, their copolymers, and combinations thereof. The layer is preferably at least about 0.001 to 50 μm thick, more preferably at least about 0.01 to 10 μm thick and most preferably at least about 0.5 μm thick.

In other embodiments, oligonucleotide probes are immobilized on polyacrylamide gel pads in contact with the microelectrodes of the apparatus of the present invention. In a preferred embodiment, the polyacrylamide gel pad is embedded into a recess etched into the surface of the microelectrode. The polyacrylamide gel pad is preferably at least about 0.1 to 30 μm thick, more preferably at least about 0.5 to 10 μm thick, and most preferably about 0.5 μm thick.

The apparatus of the present invention comprises at least one counter-electrode. In the preferred embodiment of the present invention the counter-electrode comprises a conductive material, with an exposed surface that is significantly larger than that of the individual microelectrodes. In a preferred embodiment, the counter electrode comprises platinum. In alternative embodiments, the counter electrode comprises solid or porous films of silver, gold, platinum, titanium, copper, or metal oxides, metal nitrides, metal carbides, or carbon.

In other embodiments of the present invention, the apparatus comprises at least one reference electrode. The reference electrode is used in assays where the further quantification of target molecules is desired. In preferred embodiments, the reference electrode comprises a silver/silver chloride electrode. In alternative embodiments, the reference electrode comprises solid or porous films of gold, platinum, titanium, or copper, metal oxides, metal nitrides, metal carbides, or carbon.

The electrolyte solution comprising the apparatus of the present invention is any electrolyte solution comprising at least one salt containing metal or polymerized cations that are ion-conductive and can react with biological molecules, most preferably nucleic acids or peptides. Most preferably, the salt further comprises anions that exhibit a reduced specific adsorption for the surface of the microelectrode, thereby reducing the noise during the detection of molecular interactions between probe and target molecules. In a preferred embodiment of the present invention, the electrolyte solution used for the detection of nucleic acid hybridization contains 0.1 M LiClO₄. This electrolyte is preferred since ClO₄⁻ is not specifically adsorbed on the electrode surface and thus generates a low background noise. In addition, Li⁺ is preferred since its small size facilitates intercalation of the Li⁺ cations into the nucleic acid duplex and has less diffusion resistance. However, in other embodiments, the AC impedance is measured in hybridization buffers such as 1× SSC following molecular interactions between probe and target molecules.

In the apparatus of the present invention the means for producing electrical impedance at each microelectrode can be accomplished using a model 1260 Impedance/Gain-Phase Analyser with model 1287 Electrochemical Interface (Solartron Inc., Houston, Tex.). Other electrical impedance measurement means include, but are not limited to, transient methods with AC signal perturbation superimposed upon a DC potential applied to an electrochemical cell such as AC bridge and AC voltammetry. The measurements can be conducted at certain frequency determined by scanning frequencies to ascertain the frequency producing the highest signal. The means for detecting changes in impedance at each microelectrode in the presence or absence of a target molecule can be accomplished by using one of the above-described instruments.

In still further alternative embodiments of the present invention, the apparatus further comprises a plurality of wells each of which encompasses at least one microelectrode and at least one counter-electrode. The term “wells” is used herein in its conventional sense, to describe a portion of the substrate in which the microelectrode and at least one counter-electrode are contained in a defined volume.

The present invention provides an apparatus and methods for detecting molecular interactions by detecting cation interactions associated with nucleic acid hybridization. The detection method used is most preferably AC impedance, but encompasses any detection methods that do not employ or require a reporter-labeled moiety to obtain measurable signals. The impedance is measured at different frequencies in order to obtain a “signature” of the hybridization reaction that is sensitive enough to permit mismatch hybridization between the oligonucleotide probe and target molecules to be detected. The inventive methods disclosed herein are useful for electrical detection of molecular interactions between probe molecules bound to defined regions of an ordered array (conventionally termed “a biochip array”) and target molecules in a sample which are permitted to interact with the probe molecules. By arraying microelectrodes to which individual probe molecules have been attached on a biochip, parallel measurements of many probes can be performed in a single assay.

The present invention further provides an apparatus and methods for detecting cation interactions associated with peptide binding using AC impedance, but without the use of reporter-labeled target to obtain measurable signals. The methods are used for electrical detection of molecular interactions between probe molecules bound to defined regions of an ordered peptide array and target molecules in a sample which are permitted to interact with the probe molecules. By arraying microelectrodes to which individual probe molecules have been attached on a biochip, parallel measurements of many probes can be performed in a single assay.

The apparatus and methods of the present invention can be adapted further to be used with arrays of any substance that can participate in a molecular interaction that can be interrogated with cations, most preferably lithium cations. Such interactions include ligand-receptor interactions, enzyme-inhibitor interactions, and antigen-antibody interactions.

An important advantage of the apparatus and methods of the present invention is that they are not dependent on labeling the target molecule. By removing the labeling step, the cost of the assay is reduced as well as simplified, thereby making electrical detection easier and more cost-effective to use. Furthermore, by not requiring target molecules to be labeled, the range of assays for which a method of the present invention may be employed is extended. For example, the present invention enables one to perform high sensitivity, high resolution measurements of RNA concentrations in gene expression studies without having to label the chemically-labile RNA or to convert the RNA into cDNA. The methods of the present invention may also enable new types of assays to be developed.

The preferred embodiment of the present invention and its advantages over previously investigated electrical or electrochemical detection devices are best understood by referring to FIGS. 1-14 and Examples 1-8. The Examples, which follow, are illustrative of specific embodiments of the invention, and various uses thereof. They are set forth for explanatory purposes only, and are not to be taken as limiting the invention.

### EXAMPLE 1

### Preparation of Polypyrrole Microelectrodes

Polypyrrole microelectrodes were prepared as follows. Ultra-fine platinum wire having a diameter of 50 μm was inserted into glass capillary tubing having a diameter of 2 mm and sealed by heating to form a solid microelectrode structure. The tip of the structure was then polished with gamma alumina powder (CH Instruments, Inc., Austin, Tex.) to expose a flat disk of the platinum wire. Microelectrodes were initially polished with 0.3 μm gamma alumina powder, rinsed with deionized water, and then polished with 0.005 μm powder. Following polishing, the microelectrodes were ultrasonically cleaned for 2 min. in deionized water, soaked in 1 N HNO₃ for 20 min., vigorously washed in deionized water, immersed in acetone for 10 min., and again washed vigorously in deionized water. Through the use of micromanufacturing techniques employed in the fabrication of semiconductors, modifications of this procedure can be applied to the preparation of microelectrodes of a size required for the construction of bioarray chips.

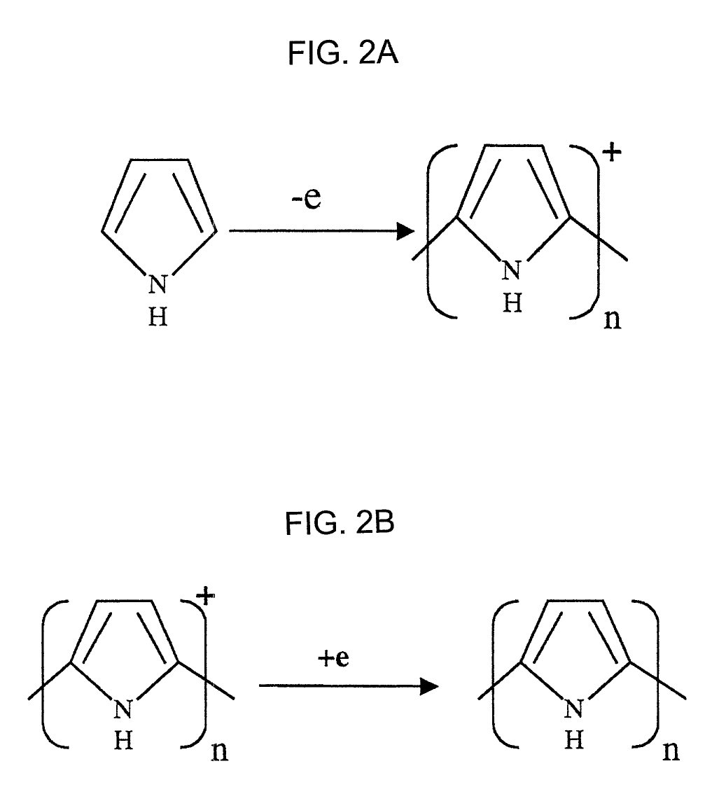

A neutral polypyrrole matrix was used for attachment of nucleic acid probes to the exposed platinum disk of the microelectrodes. Electrochemical deposition was performed using a model 660A potentiostat (CH Instruments, Inc.), using platinum wire as a counter-electrode, silver/silver chloride (Ag/AgCl) as a reference electrode, and cyclic voltammetry (CV). A solution containing 0.05 M pyrrole, 2.5 μM 3-acetate-N-hydrodysuccinimido pyrrole, and 0.1 M LiClO₄/95% acetonitrile was prepared as the electrolyte. The potential range for the CV was 0.2 to 1.3 V versus Ag/AgCl for the first cycle and −0.1 to 1.0 versus Ag/AgCl for 10 additional cycles. The scan rate was 10 mV/sec. The electrolyte was purged by nitrogen gas during the entire deposition process. Alternatively, polypyrrole film can be formed by oxidation of pyrrole at a constant current of 0.20 to 0.25 mA/cm² in the same solution described herein. This method has an advantage in the fabrication of array-based microelectrodes in that the reference electrode is not required. An electrochemical oxidation of the pyrrole produced the polypyrrole shown to the right of the arrow in FIG. 2A.

The polypyrrole electrodes in oxidized form were put into 0.1 M LiClO₄ and cycled over a potential range of −0.1 to 0.8 for 20 cycles. This procedure stabilizes the polypyrrole film. To make a neutralized polypyrrole, the microelectrodes were placed in the electrolyte again and cycled for 10 cycles over a potential range of −0.2 to 0.3 versus Ag/AgCl, which is the reduction zone for this electrochemical system. The neutralization is desired in order to reduce the background charge of the probe attachment matrix and thus increase the sensitivity of the hybridization electrical measurements. The reaction for neutralizing the polypyrrole film is illustrated in FIG. 2B. Following neutralization of the polypyrrole film coating the microelectrodes, the microelectrodes were vigorously rinsed with deionized water.

### EXAMPLE 2

### Attachment of Nucleic Acid Probes to Polypyrrole Microelectrodes

To attach nucleic acid probes to the microelectrodes prepared in Example 1, the microelectrodes were incubated at room temperature for 4 hours in a solution consisting of 80 μL dimethylformamide and 20 μL of 15 nM 5′-amino labeled 15-mer oligonucleotide (5′-C-C-C-T-C-A-A-G-C-A-G-A-G-G-A-3′; SEQ ID NO: 1). Following attachment of the probe molecules, the microelectrodes were washed with TBE buffer (0.89 M Tris-borate, 0.025 M EDTA), rinsed thoroughly in deionized water, and allowed to dry at room temperature.

### EXAMPLE 3

### Electrical Detection of Nucleic Acid Hybridization Using Polypyrrole Microelectrodes

The AC impedance baseline of the microelectrodes prepared according to Example 2 was first determined in the absence of a complementary target molecule. Microelectrodes were then exposed in a sealed conical tube to 35 μL of the complementary target molecule (5′-T-C-C-T-C-T-G-C-T-T-G-A-G-G-G-3′; SEQ ID NO: 2) present at concentrations in the micromolar (10⁻⁶ M; μM) to attomolar (10⁻¹⁸ M; αM) range. Hybridization of probe and target molecules was performed in 1× SSC buffer (0.15 M NaCl, 0.015 M sodium citrate, pH 7.0) at 37° C. for 24 to 48 hours. Following hybridization, microelectrodes were thoroughly rinsed in an excess volume of 1× SSC at room temperature and then AC impedance was measured.

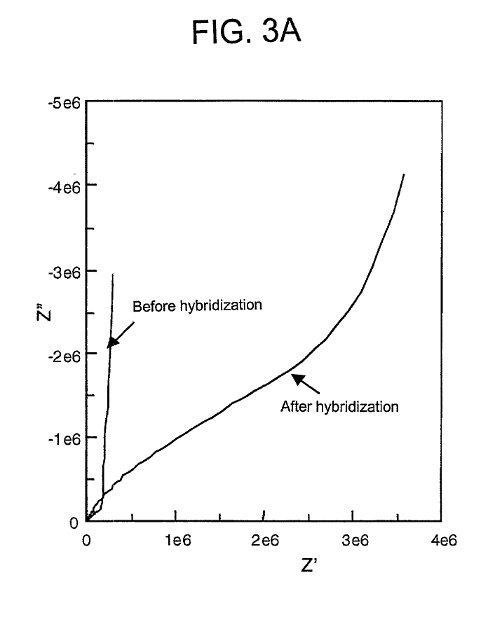

AC impedance was measured using a model 1260 Impedance/Gain-Phase Analyser with model 1287 Electrochemical Interface (Solartron Inc., Houston, Tex.). The counter and reference electrodes were platinum and Ag/AgCl, respectively, and the impedance measurements were made under open circuit voltage (OCV) conditions in a 1 M LiClO₄ solution. The measured complex impedance (Z) versus frequency for a polypyrrole microelectrode with attached 15-mer oligonucleotide before and after hybridization with a 2 ƒM solution of the complementary target molecule is shown in FIG. 3A (Complex impedance is described by the equation Z=Z′+iZ″, in which Z′ is real part of the impedance, i is √-1 and Z″ is imaginary part of the impedance). A significant difference was observed between the microelectrodes before and after hybridization to the target molecule (FIG. 3A). The large signals produced at low target concentration (i.e., 2ƒM of target is equivalent to 0.1 amol of target molecules) indicates the high sensitivity of the methods of the present invention for detecting hybridization between oligonucleotide probes and target nucleic acid molecules. The frequency increases from 0.1 Hz at large values of Z′ to 1 MHz at a Z′ of ˜0.

FIG. 3B illustrates the frequency complex curves, as seen in FIG. 3A, for the high frequency zone (where Z′<5×10⁴). Frequency dependent semicircle impedance curves were observed at high frequencies before and after hybridization. Generally, such curves at high frequencies indicate the existence of a Faraday resistance (i.e., electrochemical reaction resistance) in parallel with a capacitance. Semicircular curves such as those shown in FIG. 3B can be used to obtain the electrochemical reaction resistance and the double layer capacitance by equivalent circuit simulation. The simulation results obtained using the data shown in FIG. 3B is shown in Table I. These results indicate that following hybridization of the probe and target molecules, the high frequency electrochemical resistance decreases and the capacitance increases, which is as expected. This demonstrates that the hybridized DNA has a strong electrochemical interactions with Li⁺.

TABLE I
Equivalent Circuit Parameters Obtained from High Frequency
Impedance Data

| Nucleic Acid Status | Faraday Resistance, R (Ω) | Capacitance, C (nF) |
|---|---|---|
| Single-stranded | 2236 | 0.198 |
| Double-stranded | 922 | 0.597 |

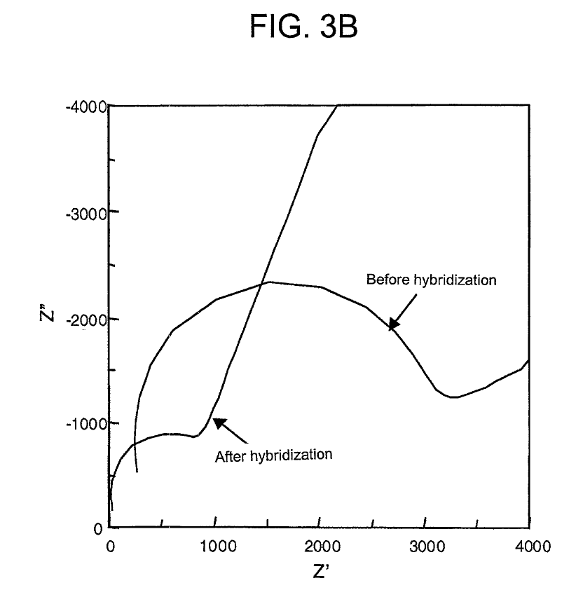

The real part of the complex impedance data shown in FIG. 3A, i.e., the resistance (R) versus the square root of the frequency (ω^{-½}), is plotted in FIGS. 4A and 4B. Linear regions are observed, demonstrating that the Li⁺ diffusion process dominates the measurements at lower frequencies. FIGS. 4A and 4B show that a significant change in the resistance occurs after hybridization of the single-stranded probe with the target molecule. The decrease in high frequency resistance following hybridization (FIG. 4B) can be explained by a decrease in the Faraday resistance of the hybridized nucleic acid. At low frequencies, the large ion diffusion resistance dominates the impedance and thus the resistance is higher for the hybridized probe-target duplex (FIG. 4A). As the frequency increases, the contribution of the frequency-dependent diffusion resistance decreases and thus the smaller Faraday resistance dominates.

The limit of detection in the experiments described above was reached at approximately 0.1 attomol of target molecule. With increased target molecule concentrations, higher hybridization signals were obtained, demonstrating that methods of the present invention can be also used to quantify the amount of target hybridized onto the electrode-immobilized probe. Thus, this method can be used in conjunction with appropriate reference electrodes to measure the absolute quantities of nucleic acid in a test sample. For example, the methods of the present invention enable one to perform high sensitivity, high resolution measurements of RNA concentrations in gene expression studies. Comparative gene expression studies performed using such a method permits the direct measurement of the quantity of expressed RNA, rather than relying on a determination of the ratio between the RNA of interest and a control RNA.

### EXAMPLE 4

### Specificity of Electrical Detection Using Polypyrrole Microelectrodes

Microelectrodes with attached oligonucleotide probes were prepared as described in Examples 1 and 2. Four microelectrodes were incubated in a solution containing 2 pM of a 1 5-mer target molecule that was fully complementary to the attached probe (5 ′-T-C-C-T-C-T-G-C-T-T-G-A-G-G-G-3′; SEQ ID NO: 2) and four other microelectrodes were incubated in a solution containing 2ƒM of a noncomplementary 15-mer target molecule (5′-C-C-C-T-C-A-A-G-C-A-G-A-G-G-A -3′; SEQ ID NO: 1) using the conditions described in Example 3.

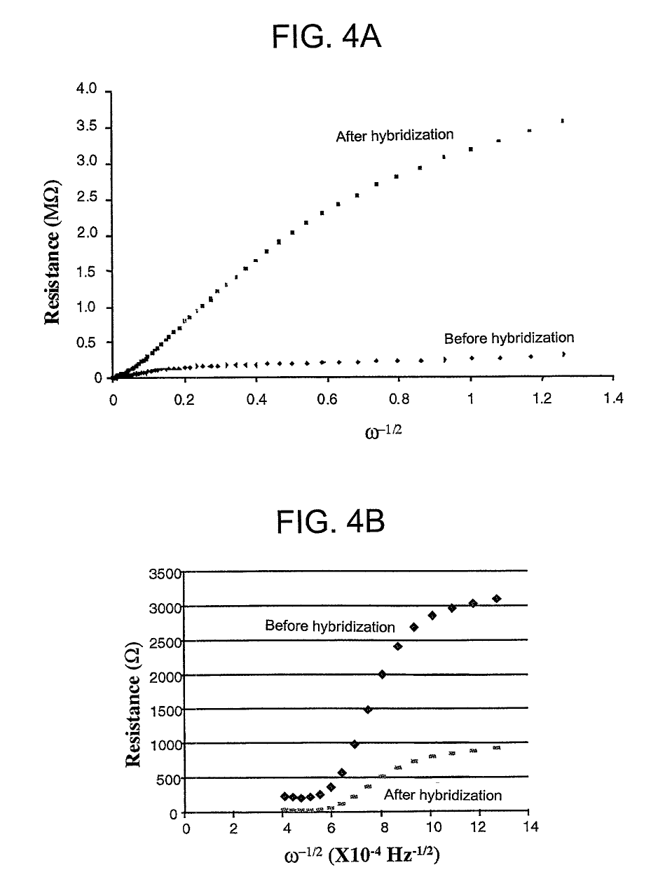

Following hybridization, individual microelectrodes were washed at successively higher temperatures to electrically measure the melting of the duplexes. Washing was performed by placing the microelectrodes in 1× SSC for 30 min. at either 37° C. or 38° C. AC impedance curves for the microelectrodes that were not hybridized, hybridized to the target, or washed at 37° C. or 38° C. are shown in FIG. 5A (fully complementary target molecule) and 5B (noncomplementary target molecule).

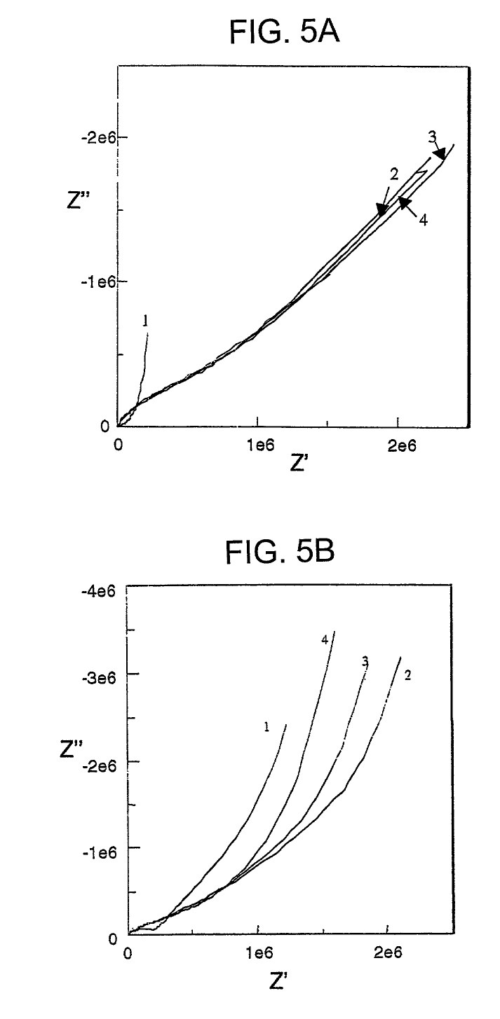

AC impedance measurements showed a pronounced difference between the fully complementary (perfect) and noncomplementary hybridized nucleic acid duplexes. The impedance curves obtained for the fully complementary target molecule remained unchanged following the washes, indicating that the melting temperature of the perfect duplex was not exceeded. The impedance curves obtained for the noncomplementary target molecule moved toward the baseline (i.e., unhybridized probe) following washes, indicating that the melting of this duplex was occurring at temperatures near to that duplexes melting temperature. The ability to discriminate between matched and noncomplementary nucleic acid sequences demonstrates the applicability of the methods of the present invention in the detection of gene polymorphism. FIG. 6 indicates that the resistance in the noncomplementary DNA system continuously decreases with increasing wash temperature going back to the baseline of the single-stranded DNA.

### EXAMPLE 5

### Use of Li⁺ Reporter in Polypyrrole Microelectrode Electrical Detection of Molecular Interactions

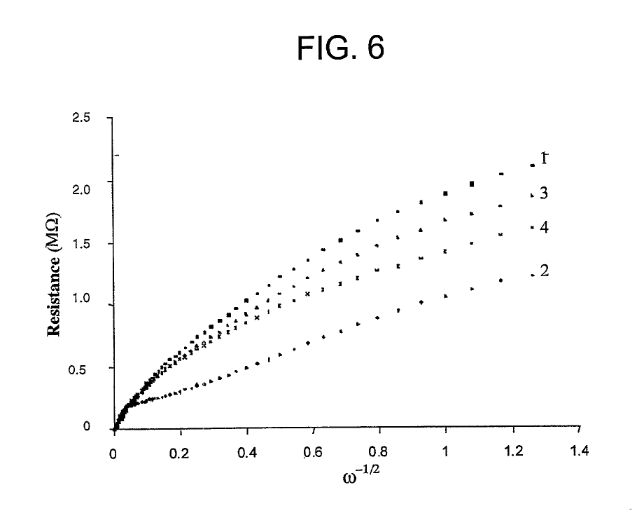

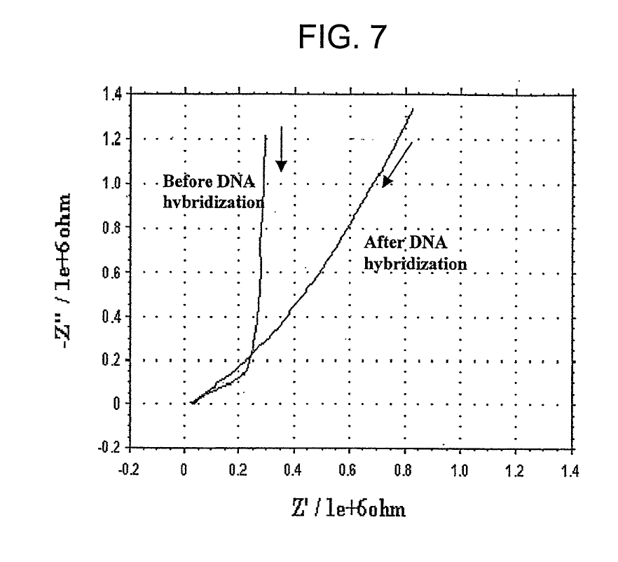

Microelectrodes were prepared as described in Examples 1 and 2 and were hybridized to suitable target molecules as described in Example 3. The AC impedance before and after nucleic acid hybridization is shown in FIG. 7. The microelectrode with attached oligonucleotide probe exhibits the characteristics of an ideal polarization electrode prior to hybridization with a target molecule. The equivalent circuit for this state is shown in FIG. 8A and the AC impedance response is shown in FIG. 8B. In this state, the microelectrode can be described by the equation: Z=Rₛ−j(ωC_{d1})⁻¹, where Z is impedance, Rₛ is solution resistance, j=√-1, ω is 2, and C_{d1} is double layer capacitance.

The behavior of the microelectrode is treated as an “ideal” polarization electrode under conditions of an electrolyte solution comprising 0.1 M LiClO₄ with purging N₂ and before hybridization to a suitable target molecule is reasonable since there is no electrochemically active species and no specific adsorption. However, following hybridization to a suitable target molecule, a large deviation from the ideal curve was observed in the same electrolyte, indicating that the impedance was significantly increased. The AC impedance measured for the microelectrode following hybridization suggests that the electrochemical process and equivalent circuit under such conditions is as shown in FIG. 8C (where Rₜ is the Faraday resistance, i.e., electrochemical reaction resistance and R_{w} is Warburg resistance). Resistance from both electrochemical reactions and the diffusion process causes the electrode behavior following hybridization to deviate from the ideal polarization curve.

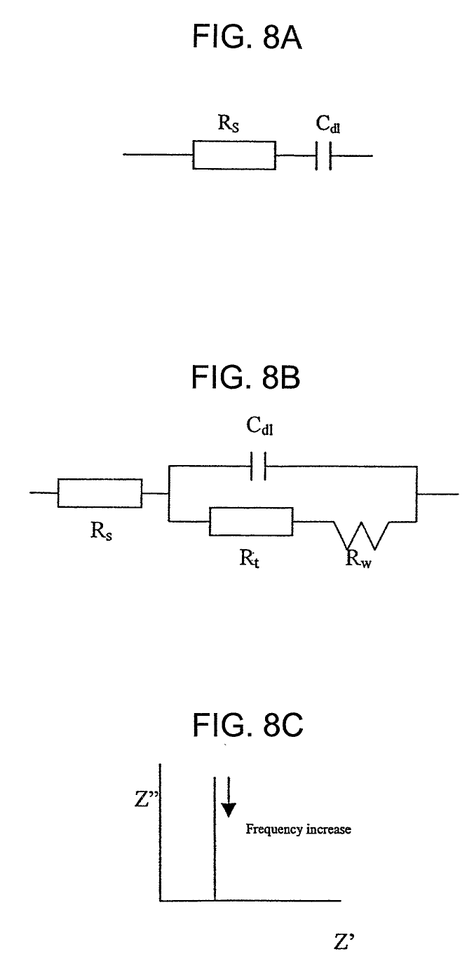

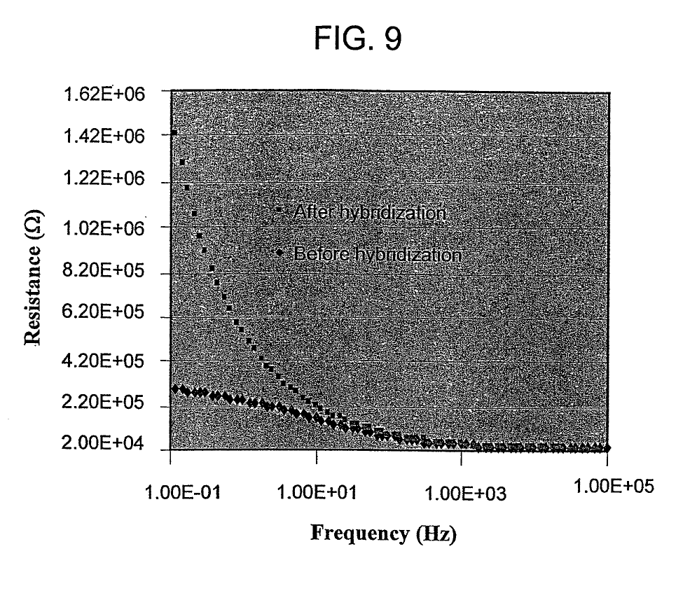

While simulation would enable the calculation of all the parameters in the equivalent circuit for this state, the equivalent circuit can also be simplified for R and C. The results of such simplification are shown in FIGS. 9 and 10, indicating that the resistance from both R and C increases as one order of magnitude. The results of the experiments described above (particularly those described in Example 4) indicate that Li⁺ in the electrolyte can serve as a reporter, permitting the mismatches between probes and target molecules to be detected. Since a method of the present invention relies on the intercalation or binding of cations, more preferably Li⁺ cations, to enable electrical detection, this method does not require that target molecules be labeled.

### EXAMPLE 6

### Preparation of Hydrogel Porous Microelectrodes

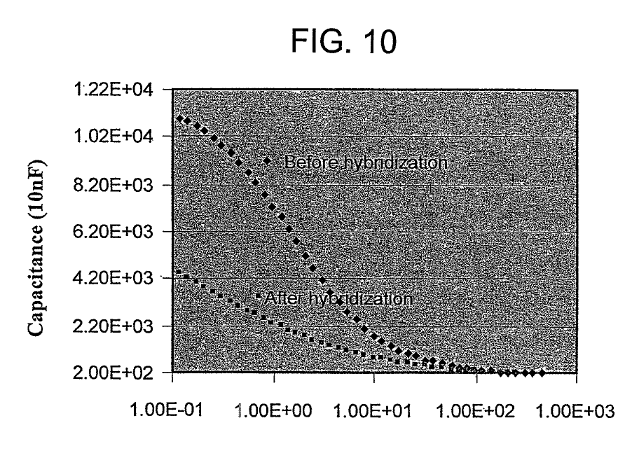

Microelectrodes were prepared as described in Example 1 (FIG. 1A). The exposed flat disk of platinum was then etched in hot aqua regia to form a recess (i.e., micropore dent) of a specified depth. The depth of the recess was controlled by the length of time that the platinum disk was exposed to the etching material. The recess thus formed was then packed with polyacrylamide gel material (FIG. 1B) to form a hydrogel porous microelectrode (FIG. 11). A hydrogel porous microelectrode having a diameter of 258 μm was used in the following Examples.

Prior to attachment of probe molecules, hydrogel porous microelectrodes were activated by incubation for 10 min. in 2% trifluoroacetic acid, and rinsed for 2 min. in deionized water. Microelectrodes were then incubated for 15 min. in 0.1 M sodium periodate, and rinsed for 2 min. in deionized water. Following this treatment, microelectrodes were thoroughly washed by incubation in deionized water for 15 min., and then air-dried. Microelectrodes were subsequently incubated for 10 min. in 2% dimethyl dichlorosilane solution and 2% octamethylcyclotetrasiloxane, washed in ethanol, rinsed in deionized water, and air-dried.

### EXAMPLE 7

### Attachment of Nucleic Acid Probes to Hydrogel Porous Microelectrodes

To attach nucleic acid probes to the microelectrodes prepared in Example 6, the microelectrodes were incubated at room temperature for 4 hours in a solution consisting of 80 μL dimethylformamide and 20 μL of 2 pM 5′-amino-3′fluorescein labeled 15-mer oligonucleotide (5′-C-C-C-T-C-A-A-G-C-A-G-A-G-G-A-3′; SEQ ID NO: 1). Following attachment of the probe molecules, the microelectrodes were washed with TBE buffer, rinsed thoroughly in deionized water, and allowed to dry at room temperature.

### EXAMPLE 8

### Electrical Detection of Nucleic Acid Hybridization Using Hydrogel Porous Microelectrodes

The baseline AC impedance of hydrogel porous microelectrodes prepared according to Example 7 was first determined in the absence of target molecules. Microelectrodes were then exposed in a sealed conical tube to either 35 μL of a complementary target molecule (5′-T-C-C-T-C-T-G-C-T-T-G-A-G-G-G-3′; SEQ ID NO: 2) present at a concentration of either 2 pM or 35 μL of a noncomplementary target molecule (5′-C-C-C-T-C-A-A-G-C-A-G-A-G-G-A-3′; SEQ ID NO: 1) present at a concentration of 300 nM. Hybridization of the probe with either target molecule was performed in 1× SSC buffer at room temperature for 1 hour. Following hybridization, microelectrodes were thoroughly rinsed for 20 min. at room temperature in an excess volume of 1× SSC and then AC impedance was measured.

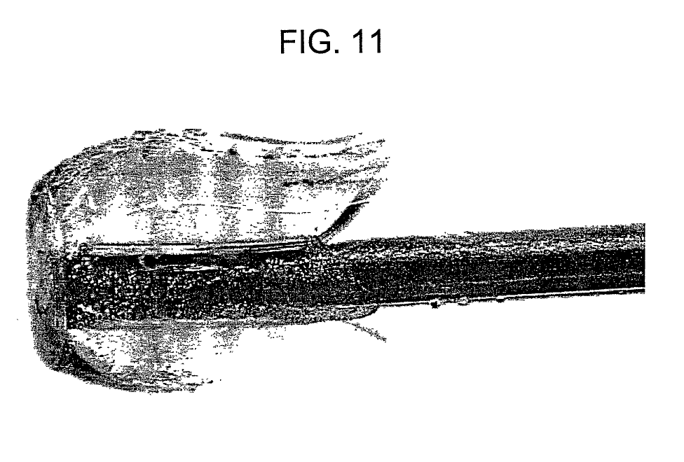

AC impedance was measured using a model 1260 Impedance/Gain-Phase Analyser with model 1287 Electrochemical Interface. The counter and reference electrodes were platinum and Ag/AgCl, respectively, and the impedance measurements were made under open circuit voltage (OCV) conditions in 1× SSC hybridization solution. Samples were excited at an amplitude of 50 mV. The measured complex impedance (Z) versus frequency for the hydrogel porous microelectrode with attached 15-mer oligonucleotide following hybridization with the complementary target molecule or noncomplementary target molecule is shown in FIG. 12.

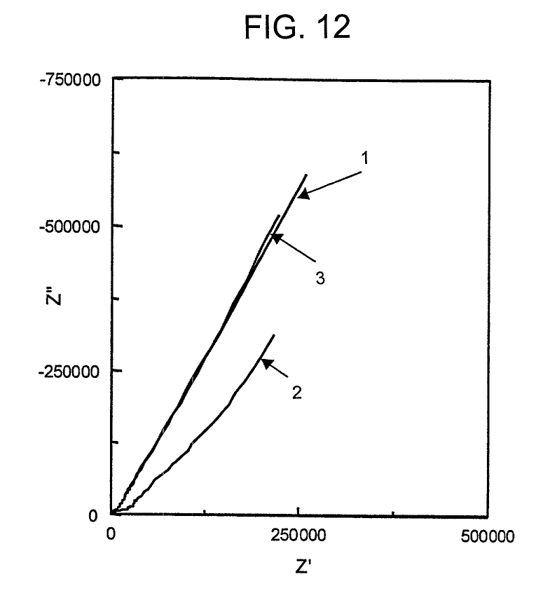

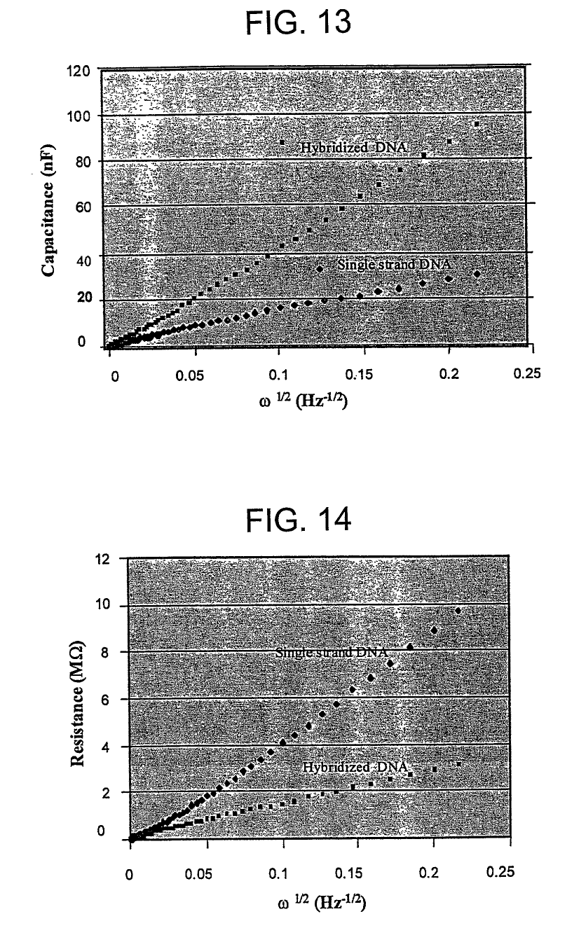

The signal generated following hybridization of probe molecules with a noncomplementary target molecule was indistinguishable from the signal generated in the absence of target molecule. The results, as shown in FIG. 12, indicate that the charge transfer has diffusion control at lower frequencies. The diffusion impedance is expressed as the Warburg element, W, and has a linear region in plots of both imaginary and real parts vs. ω^{-½}. From the imaginary and real parts of the complex impedance data shown in FIG. 12, plots of resistance (R) vs. ω^{-½} and of capacitance (C) vs. ω^{-½} were extracted and plotted as shown in FIGS. 13 and 14. Linear regions are observed in these plots, proving that a diffusion process dominates the electronic measurements. These plots show that both resistance and capacitance exhibit a significant change after the hybridization of the single stranded DNA probe with the target DNA. The resistance decreases and the capacitance increases following hybridization. These results indicate that the hybridization of target molecules to probe molecules attached to the polyacrylamide gel can improve the charge transfer process by decreasing the resistance. The increase in capacitance is due to the increase in the surface charge as a result of nucleic acid hybridization. The results obtained with the hydrogel porous microelectrode demonstrate that such microelectrodes can be used to detect 40ƒmol of target molecule in solution.

It should be understood that the foregoing disclosure emphasizes certain specific embodiments of the invention and that all modifications or alternatives equivalent thereto are within the spirit and scope of the invention as set forth in the appended claims.

What we claim is:

1. An apparatus for electrical detection of molecular interactions between an immobilized probe and a target molecule, comprising:
(a) a supporting substrate,
(b) a plurality of microelectrodes in contact with the supporting substrate to which probes are immobilized,
(c) at least one counter-electrode in contact with the supporting substrate,
(d) a means for producing electrical impedance at each microelectrode,
(e) a means for detecting changes in impedance at each microelectrode in the presence or absence of a target molecule, and
(f) an electrolyte solution in contact with the plurality of microelectrodes and the counter-electrode, wherein molecular interactions between the immobilized probe and the target molecule are detected by detecting changes in the electrical impedance in the presence and absence of the target molecule.

2. An apparatus for electrical detection of molecular interactions between an immobilized probe and a target molecule, comprising:
(a) a supporting substrate,
(b) a plurality of microelectrodes in contact with the supporting substrate,
(c) a plurality of conjugated polymer or copolymer films in contact with the microelectrodes and to which probes are immobilized,
(d) at least one counter-electrode in contact with the supporting substrate,
(e) a means for producing electrical impedance at each microelectrode,
(f) a means for detecting changes in impedance at each microelectrode in the presence or absence of a target molecule, and
(g) an electrolyte solution in contact with the plurality of microelectrodes, plurality of conjugated polymer or copolymer films, and the counter-electrode, wherein molecular interactions between the immobilized probe and the target molecule are detected by detecting changes in the electrical impedance in the presence and absence of the target molecule.

3. An apparatus for electrical detection of molecular interactions between an immobilized probe and a target molecule, comprising:
(a) a supporting substrate,
(b) a plurality of microelectrodes in contact with the supporting substrate,
(c) a plurality of polymer gel pads in contact with the microelectrodes and to which probes are immobilized,
(d) at least one counter-electrode in contact with the supporting substrate,
(e) a means for producing electrical impedance at each microelectrode,
(f) a means for detecting changes in impedance at each microelectrode in the presence or absence of a target molecule, and
(g) an electrolyte solution in contact with the plurality of microelectrodes, plurality of polyacrylamide gel pads, and the counter-electrode, wherein molecular interactions between the immobilized probe and the target molecule are detected by detecting changes in the electrical impedance in the presence and absence of the target molecule.

4. The apparatus of claims 1, 2, or 3, wherein the substrate comprises ceramic, glass, silicon, fabric, or plastic.

5. The apparatus of claims 1, 2, or 3, wherein the microelectrodes comprise a conductive material and an insulating material.

6. The apparatus of claim 5, wherein the conductive material is solid or porous gold, silver, platinum, titanium, copper, metal oxide, metal nitride, metal carbide, or graphite carbon.

7. The apparatus of claim 6, wherein the conductive material is platinum.

8. The apparatus of claim 6, wherein the conductive material is gold.

9. The apparatus of claim 5, wherein the insulating material is glass, silicon, plastic, rubber, fabric, ceramic, or combination of such materials.

10. The apparatus of claim 9, wherein the insulating material is silicon.

11. The apparatus of claim 9, wherein the insulating material is glass.

12. The apparatus of claim 5, wherein the conductive material is embedded in the substrate and the substrate comprises the insulating material.

13. The apparatus of claims 1, 2, or 3, comprising at least one reference electrode is optional.

14. The apparatus of claim 13, wherein the reference electrode comprises a conductive material and an insulating material.

15. The apparatus of claim 14, wherein the conductive material is solid or porous gold, silver, platinum, titanium, copper, metal oxide, metal nitride, metal carbide, or graphite carbon.

16. The apparatus of claim 14, wherein the conductive material is silver/silver chloride.

17. The apparatus of claim 14, wherein the insulating material is glass, silicon, plastic, rubber, fabric, ceramic, or combination of such materials.

18. The apparatus of claims 1, 2, or 3, wherein the supporting substrate further comprises a plurality of wells, each of which encompasses at least one microelectrode and at least one counter-electrode.

19. The apparatus of claim 2, wherein the conjugated polymer or copolymer film used for probe attachment includes, but is not limited to, polypyrrole, polythiphene, polyaniline, polyfuran, polypyridine, polycarbazole, polyphenylene, poly(phenylenvinylene), polyfluorene, polyindole, their derivatives, their copolymers, and combinations thereof.

20. The apparatus of claims 19, wherein probes are attached to microelectrodes using a neutral pyrrole matrix.

21. The apparatus of claims 3, wherein the gel polymer pads are polyacrylamide.

22. The apparatus of claims 1, 2, or 3, wherein the probes are oligonucleotides.

23. The apparatus of claims 1, 2, or 3, wherein the probes are nucleic acids.

24. The apparatus of claims 1, 2, or 3, wherein the probes are peptides.

25. The apparatus of claims 1, 2, or 3, wherein the electrolyte solution comprises at least one salt containing metal or polymerized cations that are ion-conductive, capable of reacting with probes or probe-target complexes.

26. The apparatus of claim 25, wherein the salt contains anions having a reduced specific adsorption for the surface of the microelectrode.

27. The apparatus of claim 25, wherein the electrolyte solution comprises 0.1M LiClO₄.

28. A method for the electrical detection of molecular interactions between an immobilized probe and a target molecule, comprising:
(a) contacting a plurality of microelectrodes to which probes have been attached with an electrolyte solution,
(b) measuring the impedance at the microelectrodes,
(c) exposing the microelectrodes to a reaction mixture containing a target molecule in order to generate probe-target complexes, and
(d) measuring the impedance at the microelectrodes.

29. The method of claim 28, wherein the electrolyte solution comprises metal, non-metal or polymerized cations that are ion-conductive and capable of reacting with probes or probe-target complexes.

30. The method of claim 28, wherein the electrolyte solution comprises 0.1 M LiClO₄ and the lithium cation is capable of reacting with probes or probe-target complexes.

31. The method of claim 28, wherein impedance is measured over a range of frequencies prior to and after exposing the microelectrodes to a reaction mixture containing the target molecule.

32. The method of claim 28, wherein impedance is measured by transient methods with AC signal perturbation superimposed upon a DC potential applied to an electrochemical cell.

33. The method of claim 28, wherein impedance is measured by impedance analyzer, lock-in amplifier, AC bridge, AC voltammetry, or combinations thereof.

34. The method of claim 28, wherein the molecular interactions detected thereby are single base mismatches within nucleic acid probe-target complexes.

35. The method of claim 28, wherein the molecular interaction detected is quantification of target molecules in a reaction mixture for gene expression analyses.
